(19) United States
(12) Patent Application Publication
(10) Pub. No.: US 2024/0050579 A1
(43) Pub. Date: Feb. 15, 2024

(54) HYALURONAN-NIMESULIDE CONJUGATE FOR TREATING LOCALLY ADVANCED OR METASTATIC TUMORS

(71) Applicant: Aihol Corporation, BUENA PARK, CA (US)

(72) Inventor: Hua-Yang LIN, Taipei City (TW)

(73) Assignee: Aihol Corporation, BUENA PARK, CA (US)

(21) Appl. No.: 18/365,528

(22) Filed: Aug. 4, 2023

Related U.S. Application Data
(60) Provisional application No. 63/370,524, filed on Aug. 5, 2022.

(51) Int. Cl. A61K 47/61; A61K 31/513; A61P 35/00

(52) U.S. Cl. CPC A61K 47/61; A61K 31/513; A61P 35/00

(57) ABSTRACT

Disclosed herein are compositions and methods for the treatment of locally advanced or metastatic tumors. According to some embodiments, the compositions and methods involve the use of a hyaluronan-nimesulide conjugate, in combination with pyrimidine-base nucleotide analog.

### CROSS-REFERENCE TO RELATED APPLICATIONS

This application claims the benefits from U.S. Provisional Application No. 63/370,524, filed Aug. 5, 2022, which is incorporated herein by reference in its entirety.

### BACKGROUND OF THE INVENTION

### 1. Field of the Invention

The present disclosure relates to compositions and methods for treating locally advanced or metastatic solid or liquid tumors in a patient. More particularly, the present compositions and methods involve the use of hyaluronan-nimesulide conjugates in the treatment of locally advanced or metastatic solid or liquid tumors.

### 2. Description of Related Art

Nimesulide is an oral non-steroidal anti-inflammatory drug (NSAID) and selective cyclooxygenase-2 (COX-2) inhibitor that has been in use an analgesic and antipyretic agent for painful musculoskeletal disorders (osteoarthritis) and primary dysmenorrhea since 1985. Since the late 1990s, numerous reports of nimesulide-induced liver injury have appeared in the medical literature. These reports have led to the withdrawal of the drug's approval in several countries and to it never being approved in others. After comprehensive evaluation of the drug's safety profile by the European Medicines Agency, the Committee for Medicinal Products for Human Use recommended restriction of nimesulide indications, withdrawal the 200 mg pharmaceutical formulation from the market, and limiting drug dosage to 100 mg twice a day. Symptoms of nimesulide-induced liver injury include jaundice, malaise and pruritus, and are generally associated with use for longer than 15 days. More recently, preclinical studies have demonstrated that nimesulide may possess anticancer activity in a number of tumor types. However, its poor solubility restricts its efficacy and limits its potential to become a leading anticancer drug.

On the other hand, cancer is a leading cause of death worldwide, accounting for nearly 10 million deaths in 2020. Patients with locally advanced or metastatic tumors often have little chance of cure.

In view of the foregoing, there exists a need in the related art to provide additional, effective compositions and methods for treating locally advanced or metastatic solid or liquid tumors.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for treating locally advanced or metastatic tumors, in a patient in need thereof.

According to some embodiments of the present disclosure, the method comprises the step of administering to the patient an effective amount of a hyaluronan-nimesulide conjugate, in combination with an effective amount of pyrimidine-based nucleotide analog, such as a fluoropyrimidine.

According to some embodiments of the present disclosure, the locally advanced or metastatic tumor may be tumors that include a locally advanced or metastatic sarcoma, pancreatic tumor, colorectal tumor, liver tumor, melanoma, lung tumor, breast tumor, ovarian tumor, head or neck tumor, stomach tumor, prostate tumor, esophageal tumor, cervical or vaginal tumor, brain tumor (such as, for example, glioblastoma, astrocytoma, meningioma, or medulloblastoma), and the like. Alternatively, the locally advanced or metastatic tumor may be liquid tumors that include a locally advanced or metastatic multiple myeloma, leukemia, or lymphoma.

In certain embodiments, the hyaluronan-nimesulide conjugate is administered to a patient at 0.1 to 3 mg/kg of nimesulide equivalents (Nim equivalents) per dose intravenously. In certain embodiments, the hyaluronan-nimesulide conjugate is administered to a patient at 5 to 150 mg/kg of hyaluronan-nimesulide conjugate per dose intravenously. In certain embodiments, the hyaluronan-nimesulide conjugate is administered on a specified interval of a treatment period. In certain embodiments, the hyaluronan-nimesulide conjugate is administered twice (on Day 1 and Day 15) of a 28-day cycle.

According to certain embodiments of the present disclosure, the fluoropyrimidine is 5-fluorouracil (5-FU), 5-fluorocytosine, 5-fluorothymidine, capecitabine, and trifluorothymidine, carmofur, doxifluridine, emitefur, tegafur, or floxuridine. In certain embodiments, the fluoropyrimidine is 5-FU. In certain embodiments, 5-FU is administered to a patient at 1 to 30 mg/kg/dose. In certain embodiments, the 5-FU is administered to the patient intravenously once weekly.

In another aspect, the present disclosure is directed to a pharmaceutical composition for treating locally advanced or metastatic tumors.

According to some embodiments, the pharmaceutical composition comprises an effective amount of a hyaluronan-nimesulide conjugate and a pharmaceutically-acceptable excipient, wherein the pharmaceutical composition is to be used in combination with a pyrimidine-base nucleotide analog, such as a fluoropyrimidine.

Subject matters that are also included in other aspects of the present disclosure include the use of a hyaluronan-nimesulide conjugate in the manufacture of a medicament for use in the treatment of locally advanced or metastatic tumors, as well as a hyaluronan-nimesulide conjugate for use in the treatment of locally advanced or metastatic tumors, wherein the medicament or the hyaluronan-nimesulide conjugate is to be used in combination with pyrimidine-base nucleotide analog, such as a fluoropyrimidine.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1A and FIG. 1B are line graphs showing the effect of co-administration of CA102N and 5-FU on cell growth of HT-29 cells, according to one working example of the present disclosure.

FIG. 2 is a line graph showing the effect of co-administration of CA102N and 5-FU on tumor volume on CT26 mice, according to one working example of the present disclosure.

FIG. 3 is a line graph showing the effect of co-administration of CA102N and 5-FU on tumor volume on CR5038 PDX mice, according to one working example of the present disclosure.

FIG. 4 is a line graph showing the effect of co-administration of CA102N and 5-FU on tumor volume in HT-29 tumor-bearing mice, according to one working example of the present disclosure.

FIG. 5 is a line graph showing the effect of co-administration of CA102N and 5-FU on tumor volume in MDA-MB-231 tumor-bearing mice, according to one working example of the present disclosure.

FIG. 6 is a bar graph showing the effect of co-administration of CA102N and 5-FU on cell growth of Bx-PC-3 cells, according to one working example of the present disclosure.

FIG. 7 is a bar graph showing the effect of co-administration of CA102N and 5-FU on cell growth of PAC-1 cells, according to one working example of the present disclosure.

FIG. 8 is a bar graph showing the effect of co-administration of CA102N and 5-FU on cell growth of A549 cells, according to one working example of the present disclosure.

FIG. 9 is a bar graph showing the effect of co-administration of CA102N and 5-FU on cell growth of MM.1S cells, according to one working example of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques. Ranges can be expressed herein as from one endpoint to another endpoint or between two endpoints. All ranges disclosed herein are inclusive of the endpoints, unless specified otherwise.

The terms “treatment” and “treating” as used herein may refer to a preventative (e.g., prophylactic), curative or palliative measure. In particular, the term “treating” as used herein refers to the application or administration of the present hyaluronan-nimesulide conjugate or a pharmaceutical composition comprising the same to a subject, who has a medical condition, a symptom associated with the medical condition, a disease or disorder secondary to the medical condition, or a predisposition toward the medical condition, with the purpose to partially or completely alleviate, ameliorate, relieve, delay onset of, inhibit progression of, reduce severity of, and/or reduce incidence of one or more symptoms or features of said particular disease, disorder, and/or condition. Treatment may be administered to a subject who does not exhibit signs of a disease, disorder, and/or condition, and/or to a subject who exhibits only early signs of a disease, disorder and/or condition, for the purpose of decreasing the risk of developing pathology associated with the disease, disorder and/or condition.

The terms “subject” and “patient” are used interchangeably herein and are intended to mean an animal including the human species that is treatable by the hyaluronan-nimesulide conjugate, pharmaceutical composition, and/or method of the present invention. The term “subject” or “patient” intended to refer to both the male and female gender unless one gender is specifically indicated.

The terms “application” and “administration” are used interchangeably herein to mean the application of a hyaluronan-nimesulide conjugate or a pharmaceutical composition of the present invention to a subject in need of a treatment thereof.

The term “effective amount” as used herein refers to the quantity of the present hyaluronan-nimesulide conjugate that is sufficient to yield a desired therapeutic response. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, as the total mass of ester prodrug (e.g., in grams, milligrams or micrograms) or a ratio of mass of ester prodrug to body mass, e.g., as milligrams per kilogram (mg/kg).

The phrase “pharmaceutically acceptable excipient” as used herein means a pharmaceutically acceptable material, composition or vehicle, such as a liquid or solid filler, diluent, carrier, solvent or encapsulating material, involved in carrying or transporting the subject agents from one organ, or portion of the body, to another organ, or portion of the body. Each excipient must be “acceptable” in the sense of being compatible with the other ingredients of the formulation. The pharmaceutical formulation contains a compound of the invention in combination with one or more pharmaceutically acceptable ingredients. The excipient can be in the form of a solid, semi-solid or liquid diluent, cream or a capsule. These pharmaceutical preparations are a further object of the invention. Usually, the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, the pharmaceutical composition of the invention is formulated into formulations suitable for the intended route of administration.

The present disclosure is based, at least in part, on the discovery that the combined use of a hyaluronan-nimesulide conjugate and a pyrimidine-base nucleotide analog (such as a fluoropyrimidine) unexpectedly achieve synergistic effect of inhibiting the growth of certain tumor in several mice models and cell lines, compared to the hyaluronan-nimesulide conjugate alone or fluoropyrimidine alone. In view of the foregoing, the present disclosure proposes methods for treating locally advanced or metastatic tumors. Some embodiments of the present disclosure are directed to methods for treating locally advanced or metastatic tumors. Also provided herein is the use of said hyaluronan-nimesulide conjugate in the treatment of said locally advanced or metastatic tumors, as well as its use in the manufacture of a medicament for said treatment purpose. The medicament (i.e., a pharmaceutical composition) is, of course, a subject matter covered by the scope of the present application.

In one aspect, the present disclosure is directed to a method for treating locally advanced or metastatic tumors, in a patient in need thereof.

According to some embodiments of the present disclosure, the method comprises the step of administering to the patient an effective amount of a hyaluronan-nimesulide conjugate, in combination with an effective amount of pyrimidine-base nucleotide analog, such as a fluoropyrimidine.

For example, the hyaluronan-nimesulide conjugate can be formulated, together with a pharmaceutically-acceptable excipient, into a pharmaceutical composition suitable for the desired mode of administration. Certain pharmaceutical compositions prepared in accordance with the presently disclosed and claimed inventive concept(s) are single unit dosage forms suitable parenteral (e.g., subcutaneous, intravenous, bolus injection, intramuscular, or intraarterial) administration to a patient. As could be appreciated, these pharmaceutical compositions are also within the scope of the present disclosure.

According to some embodiments of the present disclosure, the locally advanced or metastatic tumors may be tumors that include a locally advanced or metastatic sarcoma, pancreatic tumor, colorectal tumor, liver tumor, melanoma, lung tumor, breast tumor, ovarian tumor, head or neck tumor, stomach tumor, prostate tumor, esophageal tumor, cervical or vaginal tumor, brain tumor (such as, for example, glioblastoma, astrocytoma, meningioma, or medulloblastoma), and the like. Alternatively, the locally advanced or metastatic tumor may be liquid tumors that include a locally advanced or metastatic multiple myeloma, leukemia, or lymphoma. Additional locally advanced or metastatic solid or liquid tumors are also subject to treatment using a composition or method disclosed herein. As used herein, the term “locally advanced tumors” refers to cancer that has spread from where it started in the body to nearby tissue or lymph nodes, but not to other parts of the body. As used herein, the term “metastatic tumors” refers to cancer that has spread from where it started in the body to other parts of the body.

In certain embodiments, the hyaluronan-nimesulide conjugate is administered to a patient at 0.1 to 3.2 mg/kg of Nim equivalents per dose intravenously to yield the desired therapeutic response. In optional embodiments, the hyaluronan-nimesulide conjugate is administered to a patient at 0.15 to 2.5 mg/kg of Nim equivalents per dose intravenously; preferably, 0.2 to 2 mg/kg of Nim equivalents per dose; more preferably, 0.3 to 1.5 mg/kg of Nim equivalents per dose. Specifically, the hyaluronan-nimesulide conjugate is administered intravenously to a patient on specified days of a treatment period (or a treatment cycle) at the dose of 0.1, 0.15, 0.18, 0.2, 0.25, 0.3, 0.36, 0.4, 0.45, 0.5, 0.54, 0.55, 0.6, 0.65, 0.7, 0.72, 0.75, 0.8, 0.85, 0.9, 0.95, 1, 1.08, 1.26, 1.44, 1.5, 1.62, 1.8, 2, 2.5, 3, or 3.2 mg/kg of Nim equivalents. In certain embodiments, the hyaluronan-nimesulide conjugate is administered to mice at a dose of 2.54 to 10.16 mg/kg/dose of Nim equivalents, and thus, for a human patient, the equivalent dose is 0.205 to 0.82 mg/kg/dose of Nim equivalents.

In certain embodiments, the hyaluronan-nimesulide conjugate is administered to a patient at 5 to 150 mg/kg of hyaluronan-nimesulide conjugate per dose intravenously to yield the desired therapeutic response. In optional embodiments, the hyaluronan-nimesulide conjugate is administered to a patient at 10 to 100 mg/kg of hyaluronan-nimesulide conjugate per dose intravenously; more preferably, 15 to 50 mg/kg of hyaluronan-nimesulide conjugate per dose. Specifically, the hyaluronan-nimesulide conjugate is administered intravenously to a patient on specified days of a treatment period (or a treatment cycle) at the dose of 5, 10, 15, 20, 25, 30, 35, 40, 45, 50, 55, 60, 65, 70, 75, 80, 85, 90, 95, 100, 110, 120, 130, 140, or 150 mg/kg of hyaluronan-nimesulide conjugate. In certain embodiments, the hyaluronan-nimesulide conjugate is administered to mice at a dose of 100 to 400 mg/kg/dose of hyaluronan-nimesulide conjugate, and thus, for a human patient, the equivalent dose is 8.1 to 32.4 mg/kg/dose of hyaluronan-nimesulide conjugate.

In certain embodiments, the hyaluronan-nimesulide conjugate is administered on a specified interval of a treatment period to yield the desired therapeutic response. For example, the hyaluronan-nimesulide conjugate is administered once per month, twice per month, three times per month, every other week, once per week (QW), twice per week (BIW), three times per week (TIW), four times per week, five times per week, six times per week, every other day (QOD), daily (QD), twice a day (BID), or three times a day (TID), over a period of time ranging from about one day to about one week, from about two weeks to about four weeks, from about one month to about two months, from about two months to about four months, or from about four months to about six months, or more. In certain embodiments, the hyaluronan-nimesulide conjugate is administered twice (on Day 1 and Day 15) of a 28-day cycle at a dose of 0.18, 0.36, 0.72, 0.9, 1.08, 1.44, 2.16, 2.88 and 3.6 mg/kg of Nim equivalents.

According to certain embodiments of the present disclosure, the fluoropyrimidine is 5-FU, 5-fluorocytosine, 5-fluorothymidine, capecitabine, and trifluorothymidine, carmofur, doxifluridine, emitefur, tegafur, or floxuridine. In certain embodiments, 5-FU is administered to a patient at 1 tp 30 mg/kg/dose. In certain embodiments, 5-FU is administered to the patient intravenously once weekly. In certain embodiments, the hyaluronan-nimesulide conjugate is administered to mice at a dose of 50 to 200 mg/kg/dose of 5-FU, and thus, for a human patient, the equivalent dose is 4.05 to 16.2 mg/kg/dose of 5-FU.

In another aspect, the present disclosure is directed to a pharmaceutical composition, combined with pyrimidine, such as a fluoropyrimidine, for treating locally advanced or metastatic tumors.

According to some embodiments, the pharmaceutical composition comprises an effective amount of a hyaluronan-nimesulide conjugate and a pharmaceutically-acceptable excipient, wherein the pharmaceutical composition is to be used in combination with fluoropyrimidine.

Subject matters that are also included in other aspects of the present disclosure include the use of a hyaluronan-nimesulide conjugate in the manufacture of a medicament for use in the treatment of locally advanced or metastatic tumors, as well as a hyaluronan-nimesulide conjugate for use in the treatment of locally advanced or metastatic tumors, wherein the medicament or the hyaluronan-nimesulide conjugate is to be used in combination with pyrimidine-base nucleotide analog, such as a fluoropyrimidine.

Yet another aspect of the present disclosure is direct to the use of a hyaluronan-nimesulide conjugate in the manufacture of a medicament, combined with pyrimidine-base nucleotide analog, such as a fluoropyrimidine, for use in the treatment of locally advanced or metastatic tumors. Still another aspect of the present disclosure is direct to the use of a hyaluronan-nimesulide conjugate, in combination with pyrimidine-base nucleotide analog, such as a fluoropyrimidine, in the treatment of locally advanced or metastatic tumors. Similarly, the various dosing regimens (including dosages and dosing intervals) concerning the hyaluronan-nimesulide conjugate, pyrimidine-base nucleotide analog such as a fluoropyrimidine, and the various tumors described above are also applicable in these aspects.

The following discussion is provided to elucidate certain aspects of the present invention and to aid those of skilled in the art in practicing this invention. This discussion is in no way to be considered to limit the scope of the invention in any manner. Without further elaboration, it is believed that one skilled in the art can, based on the description herein, utilize the present invention to its fullest extent.

CA102N is a covalently bound conjugate of modified nimesulide and the sodium salt of hyaluronic acid. Specifically, CA102N is a hyaluronan conjugate having at least one disaccharide unit having the structure of,

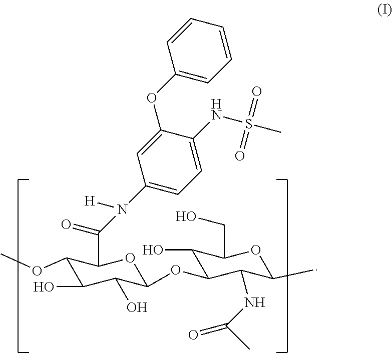

### Working Example 1

Co-Administration of CA102N and 5-FU Synergistically Inhibits Cell Growth of HT-29 Cells

In this example, HT-29 cells (colorectal adenocarcinoma) were maintained in high glucose Dulbecco's modified Eagle's medium (DMEM), supplemented with 1% penicillin-streptomycin-neomycin (PSN) antibiotic mixture, 1% L-glutamine, 1% sodium pyruvate and 10% fetal bovine serum (FBS). All culture reagents were purchase from Invitrogen. Cultures were maintained at 37° C. in a humidified atmosphere with 5% CO₂. Specifically, HT-29 cells were seeded in a 96-well plate at a density of 1×10⁴ cells/100 μL per well. 0.1 mL of varying concentrations of CA102N (equal to 0 to 400 μM of nimesulide) and/or 5-FU (0 to 200 μM) was added to each well, and the cells were cultured at 37° C. for 48 hours.

Cell viability was then determined using 3-(4,5-dimethylthiazol-2-yl)-2,5-diphenyltetrazolium bromide (MU) reduction by mitochondrial dehydrogenases. Briefly, the supernatant was removed and 100 μL of MTT solution (0.5 mg/mL in PBS) was added. After incubation for another 4 hours at 37° C., the resultant formazan crystals were dissolved in dimethyl sulfoxide (DMSO) (100 μL) and the absorbance intensity measured by scanning with multiwell ELISA reader at 570 nm (SpectraMax® M2e Multimode Plate Reader, Molecular Devices, USA). All experiments were performed in triplicate, and the data were expressed as mean SD. The relative cell viability (%) was expressed as a percentage relative to the untreated control cells.

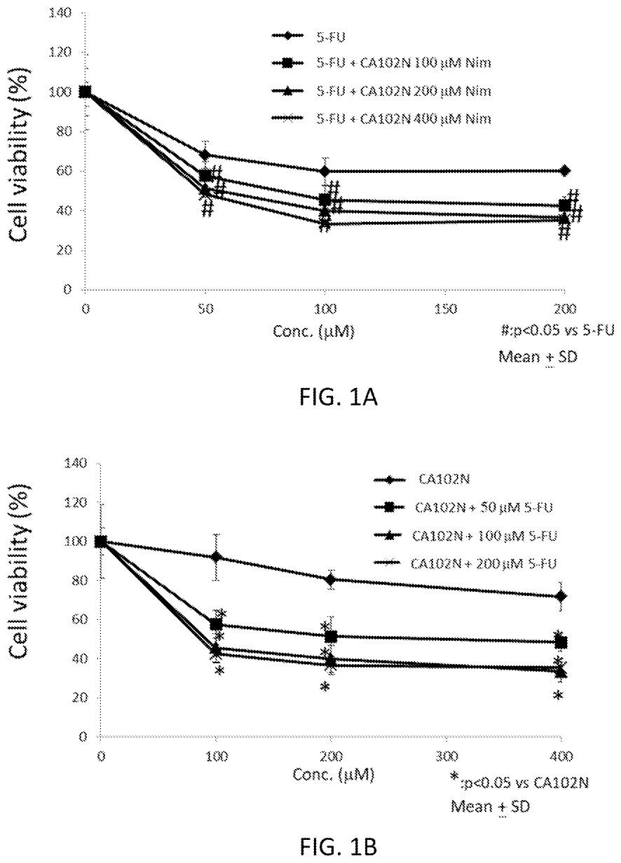

The data summarized in FIG. 1A and FIG. 1B indicate that the administration of 100, 200, or 400 μM of CA102N (equivalent of nimesulide) plus 50, 150, or 250 μM of 5-FU resulted in a reduction of the cell viability of HT-29 cells, compared to 50, 150, or 250 μM of 5-FU alone (FIG. 1A) or 100, 200, or 400 μM of CA102N alone (FIG. 1B). The effect of the co-administration of CA102N and 5-FU is in a dose-dependent manner. All co-administration groups exhibited statistically difference compared to the individual control group (p<0.05).

### Working Example 2

Co-Administration of CA102N and 5-FU Synergistically Reduces Tumor Volume in CT26 Mice

Immunocompetent, female Balb/C nude mice (ages 6-8 weeks) were ordered and received from National Laboratory Animal Center. Animals were housed on irradiated corncob bedding in micro-isolator caging. Fluorescent lighting was provided on a 12-hour cycle under temperature (20-23° C.) and humidity control (30-70%). Rodent feed and autoclaved acidified water (pH 2.5-3) were provided ad libitum.

CT26 is an N-nitroso-N-methylurethane-(NNMU) induced, undifferentiated mouse colon carcinoma cell line. CT26 carcinoma cells were grown in McCoy's 5a culture medium supplemented with 10% (v/v) FBS and 1% PSN antibiotic mixture. The cells were maintained in an incubator with a humidified environment of 5% CO² at 37° C. in a 175 cm 2 flask and subcultured 2-3 times each week. On the study day, CT26 cells were thawed and prepared for inoculation into mice. Cells were washed in PBS, counted, and resuspended in cold PBS at concentrations of 2×10⁶ viable cells/0.1 ml. Cell suspensions were mixed with an equal volume of Cultrex ECM and kept on ice during transport to the vivarium. Cells for injections were prepared by placing ECM-Cell mixture into a chilled 1 ml Lure-lok syringe fitted with a 26 7/8 G (0.5 mm×22 mm) needle. 200 μl of the cell suspension in ECM was injected subcutaneously into the rear flank. Animals were left undisturbed for seven days before observing tumor growth.

Once animals could be randomized into treatment groups, animals (n=6 each group) were treated with A) Vehicle Control (PBS), IV twice a week (BIW), B) 5-FU at 50 mg/kg, IV once a week (QW), C) CA102N at 200 mg/kg, IV twice a week (BIW), or D) CA102N at 200 mg/kg, IV twice a week (BIW)+5-FU at 50 mg/kg, IV once a week (QW).

Animals were monitored weekly for palpable tumors or any changes in appearance or behavior. Once tumors were palpable, they were measured using calipers. Tumor volume was calculated using the following equation ½(4π/3)(L/2)(W/2)H, where L is the length, W is the width, and H is the height of the tumor. Tumor Growth Inhibition (TGI) is expressed as Mean % Inhibition and was calculated by using the following formula: [1−(T−T0)/(C−C0)]*100%, where C refers to the tumor volume of the vehicle on a specific day of dosing, C0 to the tumor volume of the vehicle on the first day of dosing, T to the tumor volume of the treatment on a specific day of dosing and T0 to the tumor volume of the treatment groups on the first day of dosing. Data were expressed as mean±SEM.

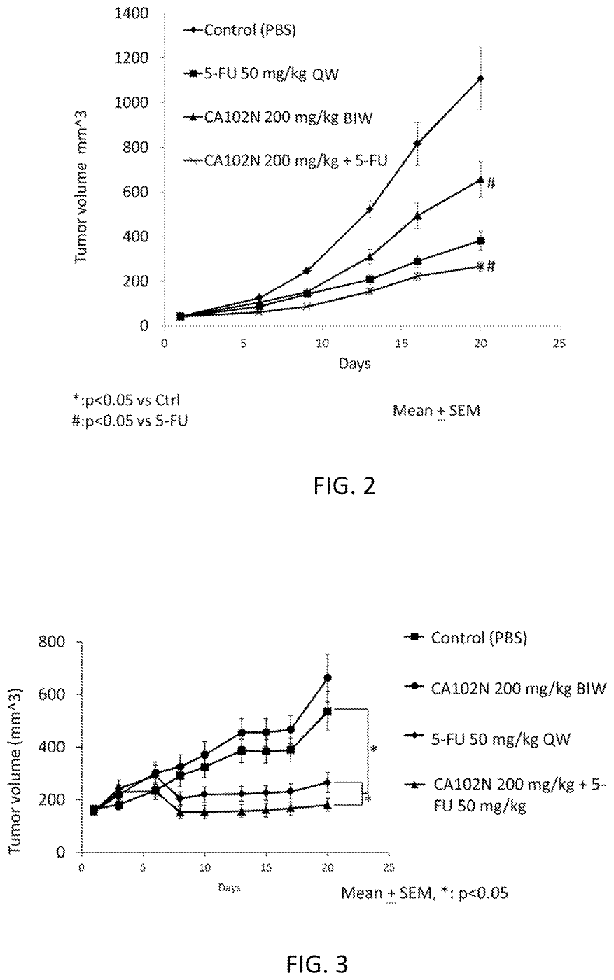

The results, as summarized in FIG. 2, indicate that the co-administration of CA102N at 200 mg/kg and 5-FU at 50 mg/kg synergistically enhanced the therapeutic efficiency, compared with CA102N alone or 5-FU alone (*: p<0.05 vs Vehicle Control; #: p<0.05 vs 5-FU). Specifically, the co-administration of CA102N and 5-FU achieved a TGI rate of approximately 80%, compared with 5-FU alone (approximately 70%), suggesting the synergic therapy efficacy of the co-administration of CA102N and 5-FU.

### Working Example 3

Co-Administration of CA102N and 5-FU Synergistically Reduces Tumor Volume in CR5038 PDX (patient derived xenograft) Mice

32 female NOD/SCID mice (ages 6-8 weeks) were purchased from Jackson Laboratories. Animals were housed in individual HEPA ventilated cages (Innocage® IVC, Innovive USA) housed for stabilization. Fluorescent lighting was provided on a 12-hour cycle under temperature (20-23° C.) and humidity control (30-70%). Rodent feed and autoclaved acidified water (pH 2.5-3) were provided ad libitum.

CR5038 PDX model is related to metastatic colon carcinoma adenocarcinoma. Tumor cryovials (CR5038), were thawed and prepared for inoculation into mice. Cells were washed in PBS, counted, and resuspended in cold PBS at concentrations of 96,000 viable cells/0.1 ml. Cell suspensions were mixed with an equal volume of Cultrex ECM and kept on ice during transport to the vivarium. Cells for injections were prepared by withdrawing ECM-Cell mixture into a chilled 1 ml Lure-lok syringe fitted with a 26 7/8 G (0.5 mm×22 mm) needle. The filled syringes were kept on ice to avoid the solidification of ECM. Animals were prepared for injection using standard approved isoflurane anesthesia and shaved prior to injection. One mouse at a time was immobilized and the site of injection was disinfected with an alcohol swab. 0.2 ml of the cell suspension in ECM was injected subcutaneously into the rear flank. Animals were left undisturbed for seven days before observing tumor growth. Animals were monitored weekly for palpable tumors, or any changes in appearance or behavior. Once tumors were palpable, they were measured using calipers. Tumor volume was calculated using the following equation (longest diameter×shortest diameter²)/2.

Once animals could be randomized into treatment groups, animals (n=8 each group) were treated with A) Vehicle Control (PBS), IV twice a week (BIW), B) 5-FU at 50 mg/kg, IV once a week (QW), C) CA102N at 200 mg/kg, IV twice a week (BIW), or D) CA102N at 200 mg/kg, IV twice a week (BIW)+5-FU at 50 mg/kg, IV once a week (QW).

The results, as summarized in FIG. 3, indicate that CA102N, alone, did not reduce tumor growth, compared to the vehicle control group. Surprisingly, the co-administration of CA102N and 5-FU significantly reduced tumor growth, compared to all other groups (*:p<0.05).

### Working Example 4

Co-Administration of CA102N and 5-FU Synergistically Reduces Tumor Volume in HT-29 Tumor-Bearing Mice

Immunocompromised, Nu/Nu, Female athymic nu/nu (nude) mice (ages 5-6 weeks, LASCO) were purchased from LASCO. Animals were housed in individual HEPA ventilated cages (Innocage® IVC, Innovive USA) housed for stabilization. Fluorescent lighting was provided on a 12-hour cycle under temperature (20-23° C.) and humidity control (30-70%). Rodent feed and water were provided ad libitum.

HT-29 human colon adenocarcinoma cells (2×10⁷) in PBS buffer were implanted subcutaneously on the right flank of each mouse. Once tumors were detectable, palpable and measurable, the primary tumor volumes were measured and recorded every 3 or 4 days. Tumor volume was calculated as ½(4π/3)(L/2)(W/2)H, where L is the length, W is the width, and H is the height of the tumor. When tumor reached an average volume of 80-100 mm³, animals were randomized into treatment and control groups and treated with a single I.V. dose. Subjects were terminated from the study when the tumor volume reached approximately 1500 mm³ or the body weight loss was greater than 20% (IACUC protocol limits).

Once animals could be randomized into treatment groups, animals (n=6 each group) were treated with A) Vehicle Control (PBS), IV three times a week (TIW), B) 5-FU at 50 mg/kg, IV once a week (QW), C) CA102N at 200 mg/kg, IV three times a week (TIW), D) CA102N at 200 mg/kg, IV once per two weeks (Q2W), E) CA102N at 400 mg/kg, IV once per two week (Q2W), F) CA102N at 400 mg/kg, IV once per week (QW), G) CA102N at 200 mg/kg, IV once per two weeks (Q2W)+5-FU at 50 mg/kg, IV once a week (QW), H) CA102N at 400 mg/kg, IV once per two weeks (Q2W)+5-FU at 50 mg/kg, IV once a week (QW), or I) CA102N at 400 mg/kg, IV once per week (QW)+5-FU at 50 mg/kg, IV once a week (QW)

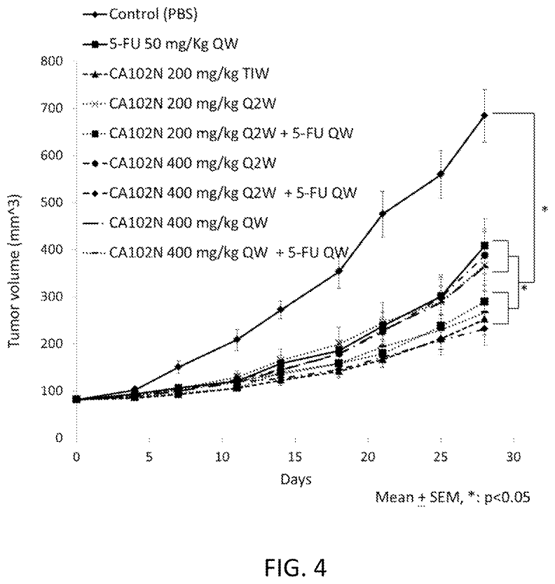

The results, as summarized in FIG. 4, indicate that 5-FU (at 50 mg/kg, QW) or CA102N (at 200 or 400 mg/kg, Q2W, or 400 mg/kg, QW), alone, did not reduce tumor growth, compared to the vehicle control group. Surprisingly, the co-administration of CA102N (at 200 or 400 mg/kg, Q2W, or 400 mg/kg, QW) and 5-FU (at 50 mg/kg, QW) significantly reduced tumor growth, compared to the control or the respective single-agent group (*: p<0.05).

Further, the TGI (tumor growth inhibition rate) in the 5-FU-treatment group showed continuous decrease from D21 to D28 (from ^{˜}60% to ^{˜}45%). Similarly, the TGI for CA102N (200 mg/kg Q2W) treatment group was ^{˜}60% on D21, ^{˜}50% on D28; the TGI for CA102N (400 mg/kg Q2W) treatment group was ^{˜}65% on D21, ^{˜}−50% on D28; the TGI for CA102N (400 mg/kg QW) treatment group was ^{˜}65% on D21, ^{˜}50% on D28. In other words, in the single-treatment groups, the TGI on the 28 days was only 50%. In contrast, the TGI for co-administration treatment groups on the 28 days is relatively higher. Specifically, the TGI for CA102N (200 mg/kg Q2W)+5-FU treatment group was ^{˜}75% on D21, ^{˜}65% on D28; the TGI for CA102N (400 mg/kg Q2W)+5-FU treatment group was ^{˜}70% on D21, ^{˜}70% on D28.

### Working Example 5

Co-Administration of CA102N and 5-FU Synergistically Reduces Tumor Volume in MDA-MB-231 Tumor-Bearing Mice

Immunocompromised, Nu/Nu, Female athymic nu/nu (nude) mice (ages 5-6 weeks, LASCO) were purchased from LASCO. Animals were housed in individual HEPA ventilated cages (Innocage® IVC, Innovive USA) housed for stabilization. Fluorescent lighting was provided on a 12-hour cycle under temperature (20-23° C.) and humidity control (30-70%). Rodent feed and water were provided ad libitum.

MDA-MB-231 human breast cancer Cells (5×10⁶) in PBS buffer were implanted subcutaneously on the right flank of each mouse. Once tumors were detectable, palpable and measurable, the primary tumor volumes were measured and recorded every 3 or 4 days. Tumor volume was calculated as ½×L×W², where L is the length and W is the width. When tumor reached an average volume of 80-100 mm³, animals were randomized into treatment and control groups and treated with a single I.V. dose. Subjects were terminated from the study when the tumor volume reached approximately 1500 mm³ or the body weight loss was greater than 20% (IACUC protocol limits).

Once animals could be randomized into treatment groups, animals (n=6 each group) were treated with A) Vehicle Control (PBS), IV three times a week (TIW), B) 5-FU at 30 mg/kg, IV three times a week (TIW), C) CA102N at 200 mg/kg, IV three times a week (TIW), D) CA102N at 400 mg/kg, IV three times a week (TIW), E) CA102N at 200 mg/kg, three times a week (TIW)+5-FU at 30 mg/kg, IV three times a week (TIW), or F) CA102N at 400 mg/kg, three times a week (TIW)+5-FU at 30 mg/kg, IV three times a week (TIW).

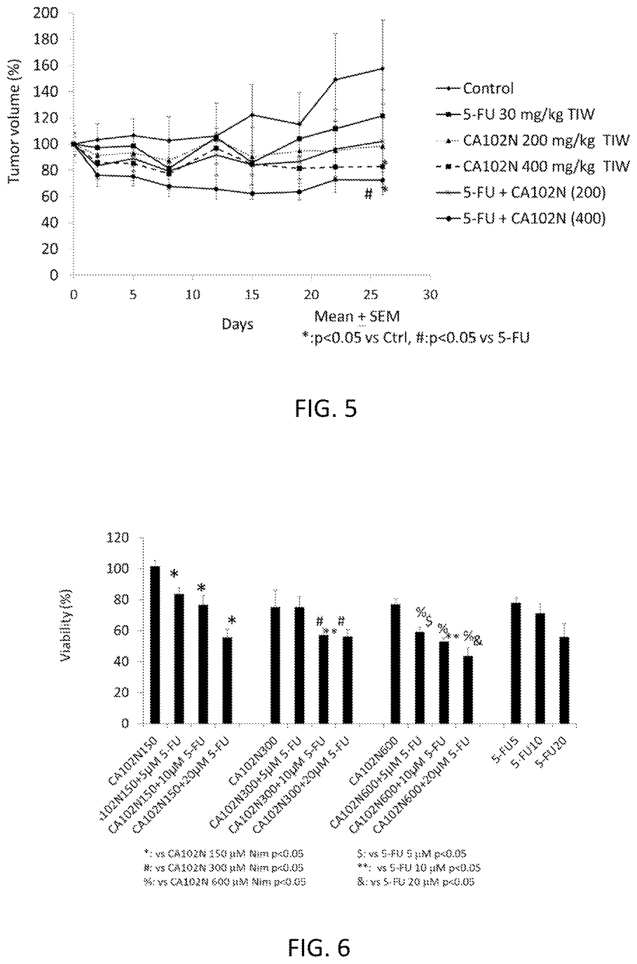

The results, as summarized in FIG. 5, indicate that 5-FU (at 30 mg/kg, TIW) or CA102N (at 200 or 400 mg/kg, TIW), alone, did reduce tumor growth, compared to the vehicle control group. However, the co-administration of CA102N (at 400 mg/kg, TIW) plus 5-FU (at 30 mg/kg, TIW) significantly reduced tumor growth, compared to the control or the 5-FU single-agent group (*: p<0.05).

### Working Example 6

### Co-Administration of CA102N and 5-FU Synergistically Inhibits Cell Growth of BxPC-3 Cells

In this example, BxPC-3 cells (human pancreatic cancer cells) were maintained in RPMI 1640 medium, supplemented with 1% PSN antibiotic mixture, 1% L-glutamine, 1% sodium pyruvate and 10% FBS. All culture reagents were purchase from Invitrogen. Cultures were maintained at 37° C. in a humidified atmosphere with 5% CO₂. Specifically, BxPC-3 cells were seeded in a 96-well plate at a density of 3×10³ cells/100 μL per well. 0.1 mL of varying concentrations of CA102N (equal to 0 to 600 μM of nimesulide) with/without 5-FU (0-20 μM) or 5-FU (5-20 μM) alone was added to each well, and the cells were cultured at 37° C. for 48 hours.

Cell viability was then determined using MTT reduction by mitochondrial dehydrogenases. Briefly, the supernatant was removed and 100 μL of MTT solution (0.5 mg/mL in PBS) was added. After incubation for another 4 hours at 37° C., the resultant formazan crystals were dissolved in DMSO (100 μL) and the absorbance intensity measured by scanning with multiwell ELISA reader at 570 nm (SpectraMax® M2e Multimode Plate Reader, Molecular Devices, USA). All experiments were performed in triplicate, and the data were expressed as mean±SD. The relative cell viability (%) was expressed as a percentage relative to the untreated control cells.

The data summarized in FIG. 6 indicate that the administration of 150, 300, or 600 μM of CA102N plus 5, 10, or 20 μM of 5-FU (except 300 μM of CA102N plus 5 μM of 5-FU) resulted in a statistically significant reduction of the cell viability of BxPC-3 cells, compared to 150, 300, or 600 μM of CA102N alone (p<0.05). The effect of the co-administration of CA102N and 5-FU is in a dose-dependent manner.

### Working Example 7

### Co-Administration of CA102N and 5-FU Synergistically Inhibits Cell Growth of PANC-1 Cells

In this example, PANC-1 cells (pancreatic cancer) were maintained in DMEM medium, supplemented with 1% PSN antibiotic mixture, 1% L-glutamine, 1% sodium pyruvate, 0.8% DMSO and 10% FBS. All culture reagents were purchase from Invitrogen. Cultures were maintained at 37° C. in a humidified atmosphere with 5% CO₂. Specifically, PANC-1 cells were seeded in a 96-well plate at a density of 5×10³ cells/100 μL per well. 0.1 mL of varying concentrations of CA102N (equal to 0 to 600 μM of nimesulide) with/without 5-FU (0-20 μM) or 5-FU (5-20 μM) alone was added to each well, and the cells were cultured at 37° C. for 72 hours.

Cell viability was then determined using MTT reduction by mitochondrial dehydrogenases. Briefly, the supernatant was removed and 100 μL of MTT solution (0.5 mg/mL in PBS) was added. After incubation for another 4 hours at 37° C., the resultant formazan crystals were dissolved in DMSO (100 μL) and the absorbance intensity measured by scanning with multiwell ELISA reader at 570 nm (Varioskan lux thermo Plate Reader, USA). All experiments were performed in triplicate, and the data were expressed as mean SD. The relative cell viability (%) was expressed as a percentage relative to the untreated control cells.

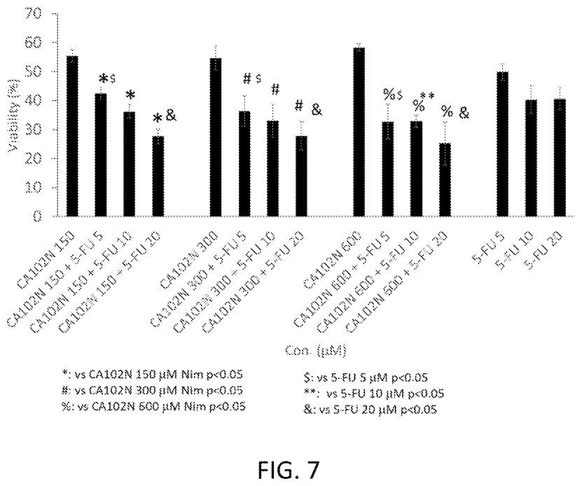

The data summarized in FIG. 7 indicate that the administration of 300 or 600 μM of CA102N plus 5, 10, or 20 μM of 5-FU resulted in a statistically significant reduction of the cell viability of PANC-1 cells, compared to 300, or 600 μM of CA102N alone (p<0.05). Further, the administration of low-dose (150 μM) CA102N plus high-dose (20 μM) 5-FU also resulted in a statistically significant reduction of the cell viability of PANC-1 cells, compared to low-dose CA102N alone (p<0.05), whereas the co-administration of low-dose CA102N and low- or medium-dose (5 or 10 μM) 5-FU also resulted in certain degrees of reduction of the cell viability of PANC-1 cells, compared to low-dose CA102N alone. The effect of the co-administration of CA102N and 5-FU is in a dose-dependent manner.

### Working Example 8

### Co-Administration of CA102N and 5-FU Synergistically Inhibits Cell Growth of A549 Cells

In this example, A549 cells (lung cancer) were maintained in F12K medium, supplemented with 1% PSN antibiotic mixture, 1% L-glutamine, 1% sodium pyruvate, 0.8% DMSO and 10% FBS. All culture reagents were purchase from Invitrogen. Cultures were maintained at 37° C. in a humidified atmosphere with 5% CO₂. Specifically, A549 cells were seeded in a 96-well plate at a density of 5×10³ cells/100 μL per well. 0.1 mL of varying concentrations of CA102N (equal to 0 to 600 μM of nimesulide) with/without 5-FU (0-20 μM) or 5-FU (5-20 μM) alone was added to each well, and the cells were cultured at 37° C. for 72 hours.

Cell viability was then determined using MTT reduction by mitochondrial dehydrogenases. Briefly, the supernatant was removed and 100 μL of MTT solution (0.5 mg/mL in PBS) was added. After incubation for another 4 hours at 37° C., the resultant formazan crystals were dissolved in DMSO (100 μL) and the absorbance intensity measured by scanning with multiwell ELISA reader at 570 nm (Varioskan lux thermo Plate Reader, USA). All experiments were performed in triplicate, and the data were expressed as mean SD. The relative cell viability (%) was expressed as a percentage relative to the untreated control cells.

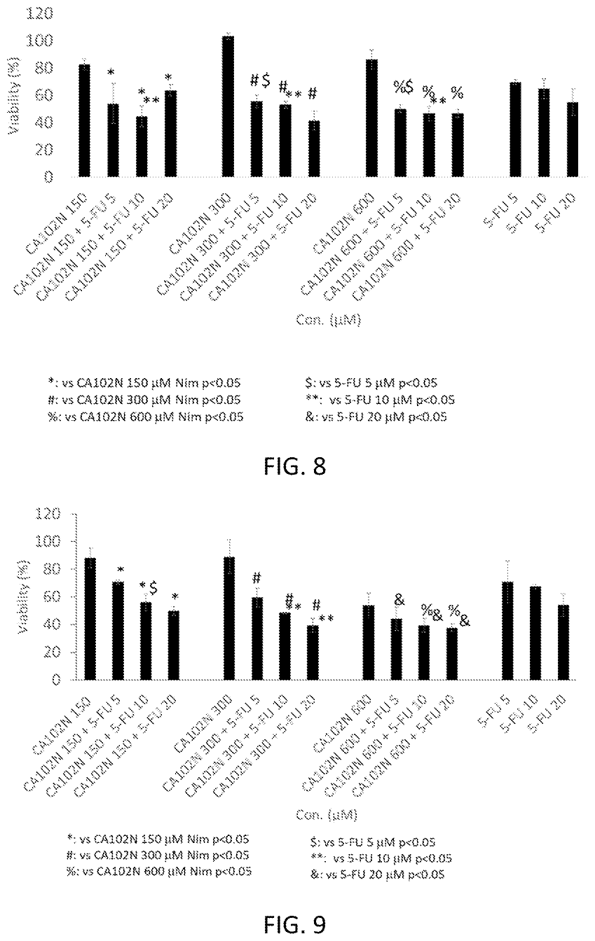

The data summarized in FIG. 8 indicate that the administration of low-dose (150 μM) CA102N plus 5, 10, or 20 μM of 5-FU resulted in a reduction of the cell viability of A549 cells, compared to low-dose CA102N alone (p<0.05). Further, the administration of medium- or high-dose (300 or 600 μM) CA102N plus high-dose (20 μM) 5-FU also resulted in a statistically significant reduction of the cell viability of A549 cells, compared to low-dose CA102N alone (p<0.05), whereas the co-administration of medium- or high-dose CA102N and low- or medium-dose (5 or 10 μM) 5-FU only reduced cell viability of A549 cells moderately, compared to medium- or high-dose CA102N alone. The effect of the co-administration of CA102N and 5-FU is in a dose-dependent manner.

### Working Example 9

### Co-Administration of CA102N and 5-FU Synergistically Inhibits Cell Growth of MM.1S Cells

In this example, MM.1S cells (multiple myeloma) were maintained in RPMI medium, supplemented with 1% PSN antibiotic mixture, 1% L-glutamine, 1% sodium pyruvate, 0.8% DMSO and 10% FBS. All culture reagents were purchase from Invitrogen. Cultures were maintained at 37° C. in a humidified atmosphere with 5% CO₂. Specifically, MM.1S cells were seeded in a 96-well plate at a density of 5×10 3 cells/90 μL per well. 90 μL of varying concentrations of CA102N (equal to 0 to 600 μM of nimesulide) with/without 5-FU (0-20 μM) or 5-FU (5-20 μM) alone was added to each well, and the cells were cultured at 37° C. for 72 hours.

Cell viability was then determined using alamar Blue assay. Briefly, after 72 hours incubation, 20 μL of alamar Blue were added to each well and then incubated at 37° C. in the dark for 4 hours. Thereafter, the plate was shaken for 1 minute and the fluorescence was detected using a fluorescence excitation wavelength of 560 nm, and the fluorescence emission was read at 590 nm by scanning with multiwell ELISA reader. All experiments were performed in triplicate, and the data were expressed as mean SD. The relative cell viability (%) was expressed as a percentage relative to the untreated control cells.

The data summarized in FIG. 9 indicate that the administration of 150, 300, or 600 μM of CA102N plus 5, 10, or 20 μM of 5-FU (except 600 μM of CA102N plus 5 μM of 5-FU) resulted in a statistically significant reduction of the cell viability of MM.S1 cells, compared to 150, 300, or 600 μM of CA102N alone (p<0.05). The effect of the co-administration of CA102N and 5-FU is in a dose-dependent manner.

It will be understood that the above description of embodiments is given by way of example only and that various modifications may be made by those with ordinary skill in the art. The above specification, examples, and data provide a complete description of the structural and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for treating a locally advanced or metastatic solid or liquid tumor, comprising administering to a patient in need thereof an effective amount of a hyaluronan-nimesulide conjugate and an effective amount of fluoropyrimidine.

2. The method of claim 1, wherein the solid tumor is a pancreatic tumor, colorectal tumor, liver tumor, melanoma, lung tumor, breast tumor, ovarian tumor, head or neck tumor, stomach tumor, prostate tumor, esophageal tumor, cervical or vaginal tumor, or brain tumor.

3. The method of claim 2, wherein the liquid tumor is multiple myeloma, leukemia, or lymphoma.

4. The method of claim 1, wherein the hyaluronan-nimesulide conjugate is administered to the patient intravenously.

5. The method of claim 4, wherein the hyaluronan-nimesulide conjugate is administered at 0.1 to 3.2 mg/kg/dose of nimesulide equivalents.

6. The method of claim 4, wherein the hyaluronan-nimesulide conjugate is administered at 5 to 150 mg/kg/dose of hyaluronan-nimesulide conjugate.

7. The method of claim 4, wherein the hyaluronan-nimesulide conjugate is administered once per month, twice per month, three times per month, every other week, once per week (QW), twice per week (BIW), three times per week (TIW), four times per week, five times per week, six times per week, every other day (QOD), daily (QD), twice a day (BID), or three times a day (TID), over a treatment period.

8. The method of claim 7, wherein the treatment period ranges from about one day to about one week, from about two weeks to about four weeks, from about one month to about two months, from about two months to about four months, or from about four months to about six months.

9. The method of claim 4, wherein the hyaluronan-nimesulide conjugate is administered twice of a treatment period of 28 days.

10. The method of claim 1, wherein the fluoropyrimidine is 5-fluorouracil, 5-fluorocytosine, 5-fluorothymidine, capecitabine, and trifluorothymidine, carmofur, doxifluridine, emitefur, tegafur, or floxuridine.

11. The method of claim 10, wherein the fluoropyrimidine is 5-fluorouracil.

12. The method of claim 11, wherein the 5-FU is administered at 1 to 30 mg/kg/dose.

13. The method of claim 11, wherein the 5-FU is administered to the patient intravenously once weekly.
